(19) United States
(12) Patent Application Publication
(10) Pub. No.: US 2016/0331220 A1
(43) Pub. Date: Nov. 17, 2016

(54) MEDICAL DEVICE CLEANING SYSTEM

(71) Applicant: COVIDIEN LP, Mansfield, MA (US)

(72) Inventors: Ricardo Gomez, Lighthouse Point, FL (US); Sandy Heck, Los Angeles, CA (US); Eric Conley, South Berwick, ME (US)

(21) Appl. No.: 15/111,005

(22) PCT Filed: Jan. 13, 2015
(86) PCT No.: PCT/US2015/011156

§ 371 Date: Jul. 12, 2016

Related U.S. Application Data
(60) Provisional application No. 61/926,753, filed on Jan. 13, 2014.

(51) Int. Cl. A61B 1/12; B08B 3/10; B08B 1/00; B08B 3/04

(52) U.S. Cl. CPC A61B 1/122; A61B 1/127; B08B 3/04; B08B 3/10; B08B 1/006

(57) ABSTRACT

A medical cleaning system is used for cleaning at least a portion of a surgical instrument. The system may include a tray, a heating pack, and a non-abrasive cleaning pouch. The tray may include a basin area used as a reservoir for maintaining, e.g., antifogging fluid, and a sloped ramp to allow liquids to flow down into its basin area. The system may also include a heating pack enclosing a heating pack solution. The heating pack solution may be a solution having nucleating properties. The heating pack solution may be activated by a mechanical activator to generate heat to the antifogging fluid. The generated heat may be transferred to the antifogging fluid residing on the non-abrasive cleaning pouch. The surgical instrument may be cleaned by wiping a portion of the surgical instrument, e.g., a lens, over the non-abrasive cleaning pouch.

### CROSS-REFERENCE TO RELATED APPLICATIONS

This application is a U.S. National Stage Application filed under 35 U.S.C. §371(a) of International Patent Application No. PCT/US2015/011156, filed Jan. 13, 2015, which claims the benefit of and priority to U.S. Provisional Patent Application No. 61/926,753, filed Jan. 13, 2014, the entire disclosure of which is incorporated by reference herein.

### BACKGROUND

1. Technical Field

The present disclosure relates to the field of cleaning medical devices and, more particularly, to a cleaning kit for cleaning medical devices used in surgery.

2. Background of Related Art

Laparoscopic devices are common medical tools used in minimally invasive surgery. Among the advantages of employing this type of surgical procedure are that large incisions, patient trauma and long recovery times are minimized. The specialized lens located at the distal end of a laparoscope has revolutionized surgery by providing the medical professional with a clear picture of the internal cavities of their patient without the need for large incisions. The laparoscope comprises a light source used in illuminating the affected area, a lens to transmit the illuminated image and an external monitor to view the transmitted image. The lens is connected to a remote monitor that allows the medical professional to clearly see and record the area being treated. When a laparoscope is inserted into the patient through a trocar/cannula device, the laparoscopic lens comes into contact with bodily fluids and matter. Debris tend to block or distort the view of the lens requiring its removal and cleaning, increasing the time needed to complete the surgical procedure.

Lens fogging is a problem that reduces patient safety, increasing procedure time and cost. It is well understood that any improvements to reduce procedural time is advantageous to the patient. There are various methods used in cleaning laparoscopic lenses, almost all require removal of the scope and cleaning with a warm antifogging fluid. Antifogging fluids are often used with non-abrasive cleaning surfaces. The problem most often encountered when using an antifogging fluid with a non-abrasive cleaning surface is the formation of air bubbles. Air bubbles distort the viewing area. Air bubbles are formed by the rubbing action of a laparoscope as it is brushed along a flat saturated cleaning surface. The air bubbles prevent the even application of the antifogging fluid to the lens necessary to provide a clearly viewable image. This further delays the surgical procedure. Once the lens area is cleaned and an even amount of antifogging fluid is applied, a clear picture of the viewing area is possible and the laparoscope can then be reinserted into the patient.

Thus, there is a need in the field for a device that better cleans medical devices, such as laparoscopic lenses. Various objects, advantages and novel features of the disclosure will be set forth in part either explicitly or implicitly in the description that follows, and in part will become apparent to those skilled in the art upon examination of the following or may be learned by practice of one or more teachings of the present disclosure. The objects and advantages may be realized and attained by means of the instrumentalities and combinations particularly pointed out in the claims that ultimately issue based on this disclosure.

### SUMMARY

A medical cleaning system used for cleaning at least a portion of a surgical instrument includes a tray adapted to accommodate cleaning fluid into which the portion of the surgical instrument may be disposed and a heating pack disposed in the tray and configured to heat the cleaning fluid.

The medical cleaning system may further include a cleaning pouch enclosing the heating pack. The cleaning pouch may be fabricated from non-abrasive material. Alternatively, the heating pack may include non-abrasive cleaning material.

The tray of the medical cleaning system may include a basin area and a sloped ramp. The sloped ramp allows the cleaning fluid, such as an antifogging fluid, to flow down into the basin area. The tray may also include a medical instrument place holder. The medical instrument place holder may be formed within a periphery edge of the tray.

The heating pack of the medical cleaning system may include a heating pack tab extending from the heating pack. The tray may include a heating pack tab opening. The heating pack tab opening may be formed within a periphery edge of the tray.

The heating pack may also include a heating pack solution therein. The heating pack solution may have nucleating properties. The heating pack may also include a mechanical activator to generate heat.

The heating pack may be secured to the tray by means of a loop and hook mechanism or other types of mechanisms.

The medical cleaning system reduces the formation of air bubbles and provides a generally evenly administered layer of antifogging fluid on the surgical instrument when the surgical instrument is wiped against the cleaning material, thus improving the visual image provided by the lens during a surgical procedure.

These and other aspects, features, and advantages of the current invention will become more readily apparent from the attached drawings and the detailed description of the preferred embodiments, which follow.

### BRIEF DESCRIPTION OF DRAWINGS

The preferred embodiments of the disclosure will hereinafter be described in conjunction with the appended drawings provided to illustrate and not to limit the disclosure, in which:

FIG. 1 is an exploded perspective view of a medical device cleaning system including a tray, a heating pack, and a cleaning pouch.

FIG. 2 is a perspective view of an embodiment of the tray of FIG. 1.

FIG. 3 is a top isometric view of the tray of FIG. 1.

FIG. 4 is a top view of the heating pack of FIG. 1.

FIG. 5 is a top isometric view of the non-abrasive heating pouch of FIG. 1.

Like reference numerals refer to like parts throughout the several views of the drawings.

### DETAILED DESCRIPTION

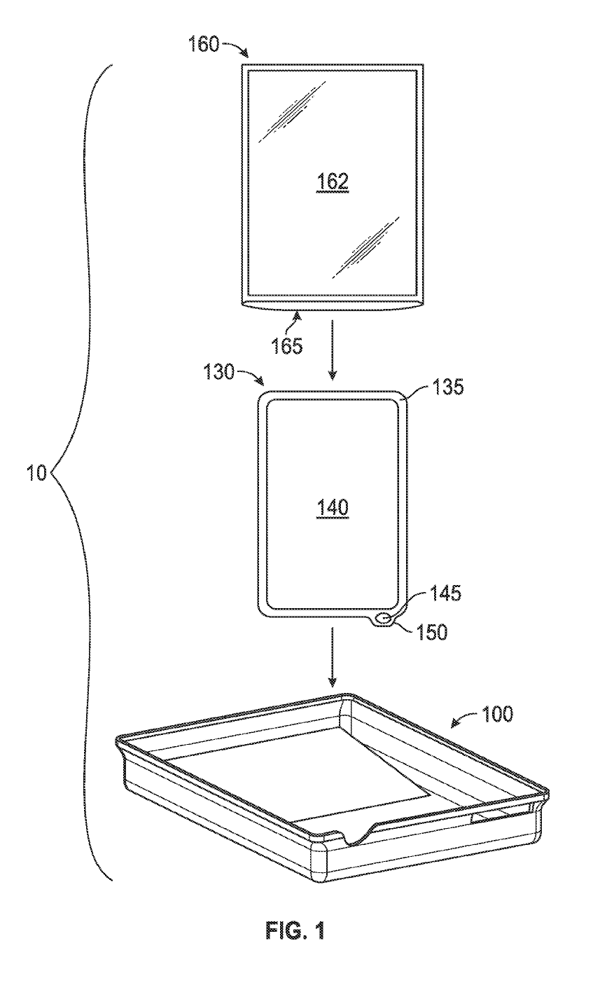

As used herein, the word “exemplary” or “illustrative” means “serving as an example, instance, or illustration.” Any implementation described herein as “exemplary” or “illustrative” is not necessarily to be construed as preferred or advantageous over other implementations. All of the embodiments disclosed in the text and figures herein are to be regarded merely as exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the disclosure as oriented in FIG. 1. Furthermore, there is no intention to be bound by any expressed or implied theory presented in the preceding technical field, background, brief summary or the following detailed description. Specific dimensions and other physical characteristics relating to the embodiments disclosed herein are not to be considered as limiting, unless the claims expressly state otherwise.

Referring to FIG. 1, a medical device cleaning system 10 includes a tray 100 adapted to accommodate a heating pack 130 therein. The medical device cleaning system 10 also may include a cleaning pouch 160 for enclosing the heating pack 130. The tray 100 is shown with its individual elements on a flat surface. The tray 100, in one embodiment, has a generally rectangular shape, but need not be limited to this rectangular shape.

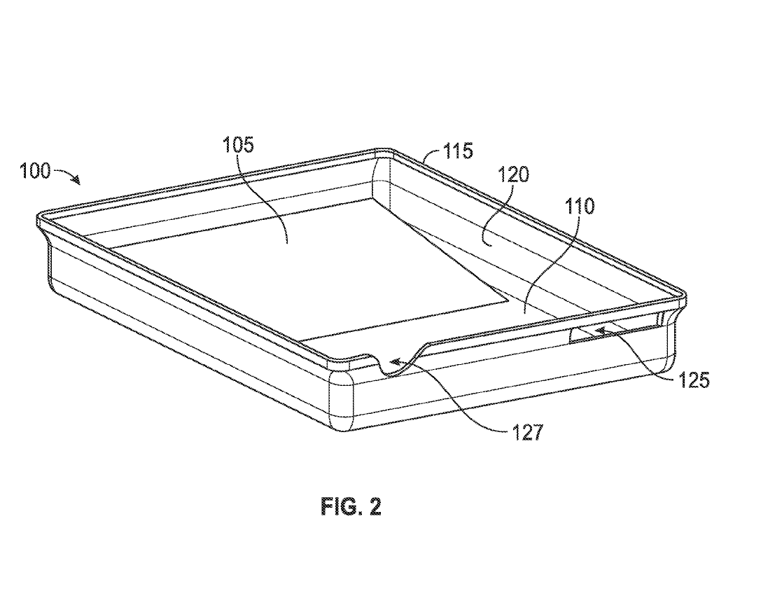

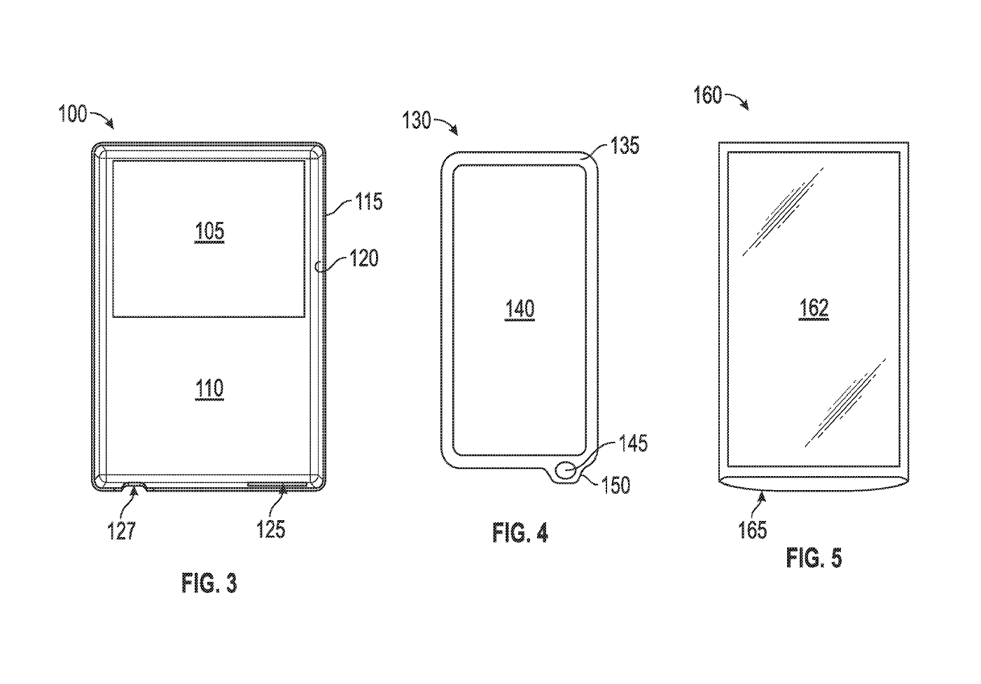

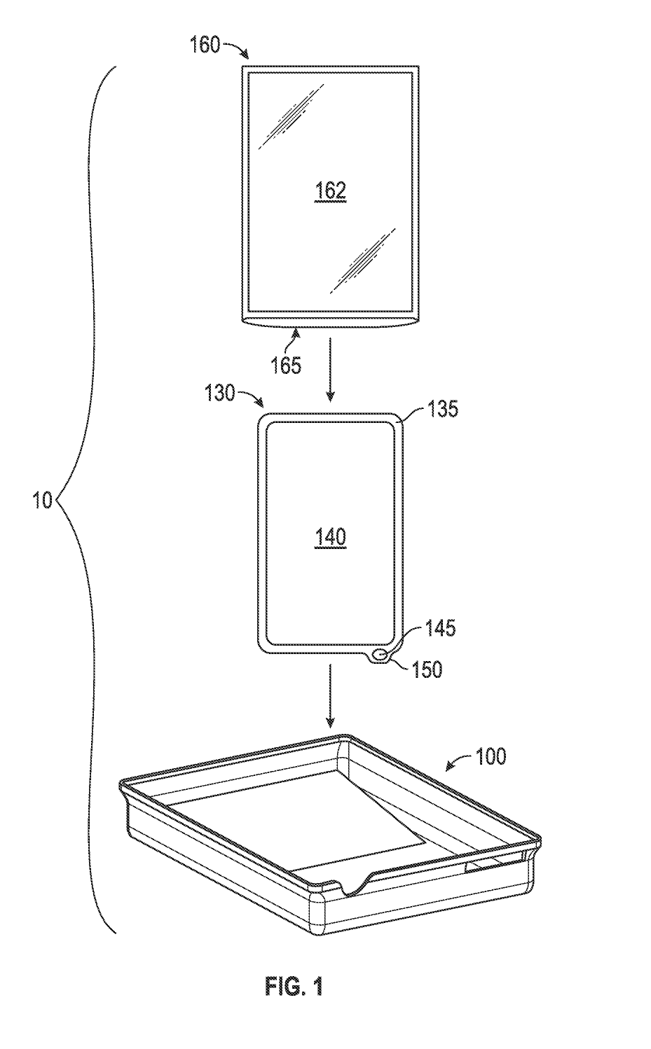

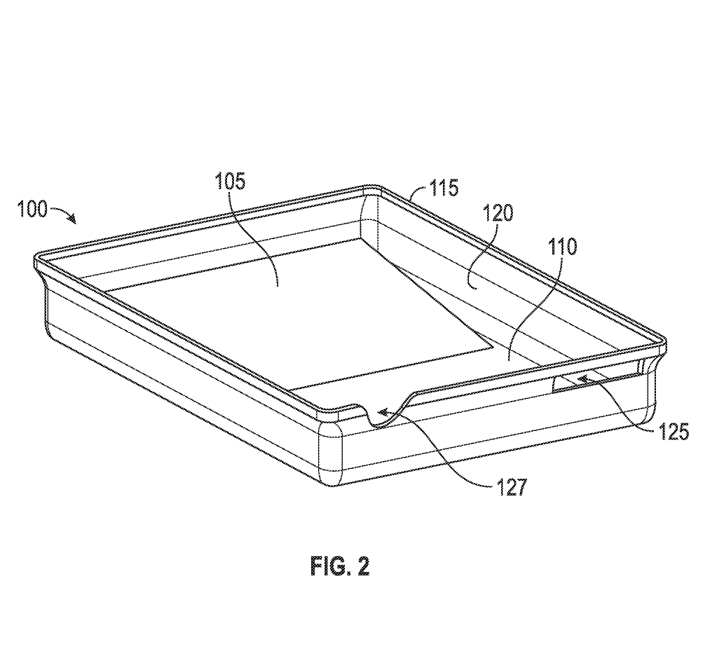

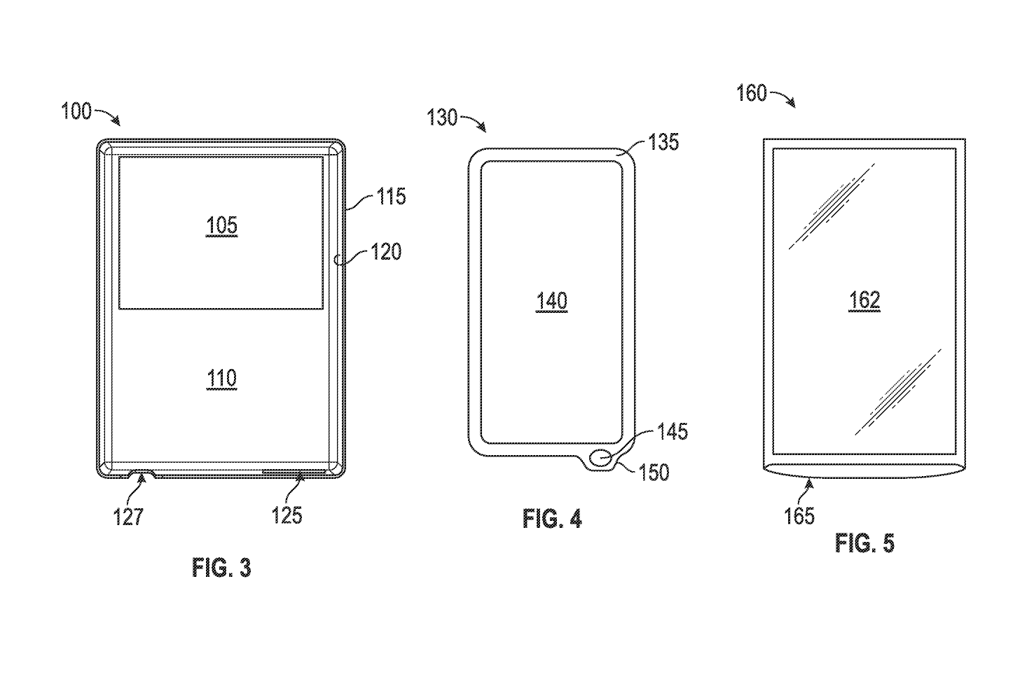

Referring to FIGS. 2 and 3, the tray 100 may include a sloped ramp 105 and basin area 1 10 with a tray periphery edge 1 15 extending therefrom. The periphery edge 1 15 includes an inner side 120 and may include a heating pack tab opening 125 and a place holder 127.

Referring to FIG. 4, the heating pack 130 may include a heating pack edge 135 surrounding the heating pack 130 with a heating pack tab 150 extending therefrom. The heating pack 130 may include heating pack solution 140 disposed therein and a mechanical activator 145.

Referring to FIG. 5, the cleaning pouch 160 may include a cleaning pouch opening 165 for accommodating the heating pack 130 therein. The cleaning pouch 160 may be fabricated from a non-abrasive cleaning material 162.

The sloped plane 105 of the tray 100, also referred to previously as the sloped ramp 105, is used to permit excess fluids to return to the basin area 1 10. Antifogging fluids (not shown) are contained within the basin area 1 10. The periphery edge 1 15 helps to maintain the heating pack 130 and non-abrasive cleaning pouch 160 of FIGS. 4 and 5, respectively, within the tray 100 structure. The tray 100 may have an opening at its frontal plane in the form of a small rectangle, referred to previously as the heating pack tab opening 125. This opening may allow the heating pack tab 150 to extend outside the tray 100 so that the user can manually activate the warming properties of the heating pack 130. The heating pack tab opening 125 is placed high enough so that any fluids that are stored in the basin area 1 10 are prevented from flowing out or do not easily flow out.

Referring back to FIG. 3, a top view of an embodiment is shown prior to adding any fluids (not shown) or the introduction of the heating pack 130, or the non-abrasive cleaning pouch 160. The place holder 127 helps to keep the medical instrument in place during use. In another embodiment, not shown, the place holder 127 has the shape of the medical instrument that is being cleaned. The heating pack tab opening 125 can also have different shapes and sizes to accommodate the mechanical activator 145 of the heating pack 130 such that the mechanical activator 145 may extend outside of the tray 100.

Referring back to FIG. 4, the heating pack 130 is shown separately from the medical device cleaning system, also referred to herein as a heated antifogging liquid applicator system 10. In one embodiment, a thick transparent and heat transferring material may be used to visually inspect the heating pack solution 140 and to prevent any leaking caused by small punctures. In an embodiment, the heating pack solution 140 may be composed of solutions having nucleating properties activated by the mechanical activator 145 to generate heat. A well sealed heating pack edge 135 may surround the heating pack 130 periphery, so as to prevent leaking. It should be understood that the disclosure is not limited to solutions having nucleating properties alone, and other heat-generating methods may also be employed.

Referring back to FIG. 5, the non-abrasive cleaning pouch 160 may be used to enclose the heating pack 130 and to transfer the heat generated therefrom to the antifogging fluid (not shown). The cleaning pouch opening 165 at one of the edges of the non-abrasive cleaning pouch 160 may be used to insert heating pack 130. The non-abrasive material 162 may be used as the main material for the pouch 160, and can be in the form of a non-abrasive fabric, cloth, foam, plastic or any other commercially acceptable material.

Referring back to FIG. 1, the non-abrasive cleaning pouch 160 may be placed over the heating pack 130 and inserted into tray 100, whereby the heating pack tab 150 may extend beyond the heating pack tab opening 125 of the tray 100. Fluids not limited to but including antifogging solutions may be used to fill the basin area 1 10. The fluids will remain below the heating pack tab opening 125. When the system is ready for use, the mechanical activator 145 may be triggered, thereby generating kinetic energy to the nucleating heating pack solution 140. The heating pack 130 may heat the antifogging solution. It should be noted that the antifogging liquid applicator system 10 may also come in a sterile self-contained enclosure, thus keeping the components sterile until usage. Any or all of the components may also be individually sterilized and packaged prior to usage.

In another embodiment, the heating pack 130 may incorporate the non-abrasive cleaning material, thereby eliminating the need for a separate cleaning pouch 160.

In another embodiment, not shown, an adhesive material or hook and loop fastener or attachment means may be used to maintain the non-abrasive cleaning pouch 160 in position relative to the sloped ramp 105 of the tray 100.

The present disclosure, according to various embodiments, provides a portable heated antifogging system, used in preventing the formation of air bubbles during cleaning of various medical instruments, including, for example, laparoscopic devices. The cleaning means can include specially designed non-abrasive fabrics, sponges, foams or any other absorbing materials. Such cleaning means may be used to clean, for example, a distal end of a laparoscopic lens or other medical device by applying an even layer of heated antifogging fluid.

The medical device cleaning system 10 of the present disclosure defogs medical lenses and helps to reduce the associated problem of air bubbles forming at the cleaning surface. Although certain embodiments may contain a flat basin, providing a sloped ramp 105 in the tray 100 may prevent the fluid from forming air bubbles when the medical device lens is rubbed repeatedly. By eliminating the formation of air bubbles, the lens surface of the medical device is allowed to acquire a smooth evenly administered layer of antifogging fluid.

It should be noted that the heating pack 130 is not limited to having nucleating properties, but may also employ exothermic, electrical, chemical or other means to provide the necessary heating to the antifogging fluid. In other embodiments, the heated antifogging liquid applicator 10 may incorporate a holding device for laparoscopes that helps position a lens of a laparoscope or medical device in a particular holding position for being heated.

In another embodiment, suction cups, mechanical means or adhesive materials may also be used at the base of the tray 100 to hold it in place when in use.

As will be recognized by those of ordinary skill in the pertinent art, numerous modifications and substitutions can be made to the above-described embodiments of the present disclosure without departing from the scope of the disclosure. Accordingly, the preceding portion of this specification is to be taken in an illustrative, as opposed to a limiting, sense.

What is claimed is:

1. A medical cleaning system used for cleaning at least a portion of a surgical instrument comprising:
a tray adapted to accommodate cleaning fluid into which the portion of the surgical instrument may be disposed; and
a heating pack disposed in the tray and configured to heat the cleaning fluid.

2. The medical cleaning system according to claim 1, further comprising a cleaning pouch enclosing the heating pack.

3. The medical cleaning system according to claim 2, wherein the cleaning pouch is non-abrasive.

4. The medical cleaning system according to claim 1, wherein the heating pack includes non-abrasive cleaning material.

5. The medical cleaning system according to claim 1, wherein the tray includes a basin area and a sloped ramp.

6. The medical cleaning system according to claim 5, wherein the sloped ramp allows the cleaning fluid, such as an antifogging fluid, to flow down into the basin area.

7. The medical cleaning system according to claim 1, wherein the tray includes a medical instrument place holder.

8. The medical cleaning system according to claim 7, wherein the medical instrument place holder is formed within a periphery edge of the tray.

9. The medical cleaning system according to claim 1, wherein the heating pack includes a heating pack tab extending from the heating pack.

10. The medical cleaning system according to claim 9, wherein the tray includes a heating pack tab opening.

11. The medical cleaning system according to claim 10, wherein the heating pack tab opening is formed within a periphery edge of the tray.

12. The medical cleaning system according to claim 1, wherein the heating pack includes a heating pack solution therein.

13. The medical cleaning system according to claim 12, wherein the heating pack solution has nucleating properties.

14. The medical cleaning system according to claim 1, wherein the heating pack includes a mechanical activator to generate heat.

15. The medical cleaning system according to claim 1, wherein the heating pack is secured to the tray.

16. The medical cleaning system according to claim 15, wherein the heating pack is secured to the tray by means of a loop and hook mechanism.

17. A medical cleaning system used for cleaning at least a portion of a surgical instrument's lens comprising:
a tray adapted for housing an antifogging fluid therein;
a heating pack configured to be placed in the tray; and
a cleaning pouch enclosing the heating pack, wherein the heating pack warms the antifogging fluid;
wherein the surgical instrument may be wiped against the cleaning pouch.

18. The medical cleaning system according to claim 17, wherein the tray includes a basin area and a sloped ramp, wherein the sloped ramp allows the antifogging fluid to flow down into the basin area.

19. A medical cleaning system used for cleaning at least a portion of a lens of a surgical instrument, comprising:
a tray having a basin area and a sloped ramp, wherein the sloped ramp allows an antifogging fluid to flow down into the basin area; and
a heating pack placed in the tray, the heating pack having a cleaning material thereon;
wherein the heating pack warms the antifogging fluid; and
wherein the cleaning material is composed of a material that reduces the formation of air bubbles, and provides a generally evenly administered layer of antifogging fluid, on the surgical instrument when the surgical instrument is wiped against the cleaning material, thus improving the visual image provided by the lens during a surgical procedure.

20. The medical cleaning system according to claim 19, wherein the cleaning material is non-abrasive.
